(19) United States
(12) Patent Application Publication
(10) Pub. No.: US 2010/0040701 A1
(43) Pub. Date: Feb. 18, 2010

(54) COMPOUND FOR EXFOLIATING SKIN

(76) Inventor: Neal Schwartz, Ellicott City, MD (US)

Correspondence Address: THE LAW OFFICES OF ANTHONY L. MEOLA, Suite 401, 2500 Westchester Avenue, Purchase, NY, 10577, US

(21) Appl. No.: 12/538,212

(22) Filed: Aug. 10, 2009

Related U.S. Application Data
(60) Provisional application No. 61/088,147, filed on Aug. 12, 2008.

(51) Int. Cl. A61K 8/26; A61K 8/19; A61K 8/23; A61K 8/97; A61K 8/362; A61K 8/365; A61K 8/49; A61Q 19/00

(52) U.S. Cl. 424/691; 514/770; 424/705; 424/725; 424/773; 514/574; 514/557; 514/184; 424/765

(57) ABSTRACT

A novel exfoliating compound and method for treating acne without an irritating cleanser. The compound includes a first subcompound formed from a clay based substance and a second subcompound formed an exfoliating substance selected from the group consisting of crystals, beads, polyethylene, nuts, natural rough products, corundum crystals, crushed almond shells and crushed apricot kernels.

### CROSS REFERENCE TO RELATED APPLICATIONS

The present application relates to and takes priority from co-pending U.S. Provisional Application Ser. No. 61/088,147 filed on Aug. 12, 2008 and entitled “A COMPOUND FOR EXFOLIATING SKIN”, which application is hereby incorporated by reference its entirety.

The present application relates to and takes priority from co-pending U.S. Non-Provisional application Ser. No. 12/489,088, filed on Jun. 10, 2009 and entitled “A COMPOUND AND METHOD FOR TREATING ACNE”, which application is hereby incorporated by reference its entirety.

### BACKGROUND OF THE INVENTION

1. Field of the Present Invention

The present invention relates generally to a compound for exfoliating skin, and in particular a compound for exfoliating skin with a clay base and non irritating exfoliating crystal like structures.

2. Description of the Related Art

The skin of the human body periodically requires deep cleansing in order to remove sebaceous secretions, soil deposits and dead skin resulting from desquamation of the epidermal tissue. Exfoliation can be accomplished by the use of a rough cleaning rag, a natural sponge or an exfoliant composition.

Known exfoliant compositions comprise an abrasive component to facilitate removal of the above-mentioned substances and debris. While such compositions are generally applied to the skin for cosmetic purposes, their use as paint removers (for use, for instance, after decorating) and in methods of medical treatment, such as treatment of acne, has also been proposed. It has been suggested that exfoliative cleaning promotes regeneration of the epidermal tissues such that the skin regains suppleness. It has also been proposed that the penetration of cosmetic or dermo-pharmaceutical products is facilitated by exfoliation.

Known exfoliant abrasives may include hydrogenated fats, inorganic salts such as sodium citrate or relatively low-molecular weight organics, such as sugars (U.S. Pat. No. 4,048,123); synthetic polymers such as polyethylene powders and granulated particles (JP 60-152407) or organo-polysiloxane (EP 295886); vegetable matter such as the endocarp of apricot, peach and walnut seeds, almond flesh, and wood flour (U.S. Pat. No. 4,508,634). Traditionally, birch powder or coconut pulp have been suggested as exfoliants. Other proposals have related to the use of animal matter such as pearl dust (JP 56-131512) or powdered crab shell and finely ground minerals such as, silica or 10 micron glacial limestone of a highly specific type (EP 257458).

Mineral exfoliants also include those listed in the patent of Saperstein (U.S. Pat. No. 3,092,111) wherein the use of aluminium oxide, synthetic alumina, corundum, volcanic ash, diatomaceous earth, bentonite, feldspar and silica is suggested. Known exfoliant compositions are provided as thick pastes for topical administration to small regions of the body such as the face or feet.

In addition, known exfoliants have also been combined with a cleanser in some treatments of acne. However, mixed results have occurred in particular with individuals having sensitive skin. The combination of an exfoliants and a cleanser can lead to additional skin irritation and eventually a reoccurring problem with skin color, appearance and general healthiness.

For some patients, anti-acne medications can be too strong because acne patients tend to be overly-sensitive and ironically are most in need of an exfoliation and oil absorption product.

Therefore, there exists a need for an exfoliant compound that provides the benefit of cleaning the skin without irritation. Such an exfoliant will likely not include a cleanser in order to benefit individuals with sensitive skin, such as those persons suffering from acne.

### SUMMARY OF THE INVENTION

In a first aspect the present invention encompasses an exfoliating compound and treating acne without an irritating cleanser. The compound includes a first subcompound formed from a clay based substance and a second subcompound formed an exfoliating substance selected from the group consisting of crystals, beads, polyethylene, nuts, natural rough products, corundum crystals, crushed almond shells and crushed apricot kernels.

In some embodiments of the present invention the compound includes 10% sulfur. In some embodiments of the present invention the compound contains various percentages of deionized water, kaolin, bentonite, glycerin, zinc oxide, eucalyptus oil, propylene glycol, magnesium aluminum silicate, polysorbate-20, titanium dioxide, diazolidinyl urea, and propylparaben.

In still other embodiments of the present invention the compound includes less than 1 percent of Sclerotium Gum, Sisymbrium Officinale Extract, Actium Lappa Root Extract, Citric Acid, Glycolic Acid, and Zinc PCA.

In some other embodiments of the present invention the compound includes various percentages of corundum #80, strawberry achenes (300-500 um) or Raspberry Seeds (300-500 um).

In some other embodiments of the present invention the compound includes a pumice element in an amount of about 25 to about 35% of the whole compound.

### DETAILED DESCRIPTION OF THE PREFERRED EMBODIMENTS

The present disclosure will now be described more fully in which an embodiment of the present disclosure is described. The subject matter of this disclosure may, however, be embodied in many different forms and should not be construed as being limited to the embodiments set forth herein.

The idea of this invention is to provide clay based exfoliation compound without irritating the skin of the user. The ideal user of the compound is one who, due to acne, suffers from highly sensitive skin. Typically these highly sensitive users are in the most need of oil absorption and exfoliation. The clay based ingredient is sufficiently viscous such the user may apply the compound to the skin in a fashion similar to that of a lotion. The compound and the preferred method of use is specifically engineered to avoid being dried out upon the user's skin. Such drying out can cause significant irritation, especially for the highly sensitive user.

The compound includes a clay base substance primarily for oil absorption. An exfoliation ingredient is added in order to remove dirt and dry skin from the surface of the patient's skin. In some embodiments a medicated drying antibacterial agent is added to the composition to destroy bacterial. In some embodiments a medicated anti acne element is added to the composition in order to combat acne. Typically this anti acne ingredient is sulphur or benzoyl peroxide and can be in amount of about 2% to about 10%.

The option to use a medicated ingredient in order to help prevent acne or destroy bacteria is dependent upon the skin sensitivity of the user. The clay based substance can perform the requisite oil absorbing with or without the anti acne medication.

The clay based substance may include all, or some of the following in various percentages: deionized water, kaolin, bentonite, glycerin, zinc oxide, eucalyptus oil, propylene glycol, magnesium aluminum silicate, polysorbate-20, titanium dioxide, diazolidinyl urea, and propylparaben. Preferably the clay based substance has a higher viscosity than standard clays such that the clay based substance moves more like a lotion which also helps prevent drying out of the clay based substance.

While some embodiments and include a soap ingredient, most acne sufferers will not be able to tolerate a soap ingredient. Therefore, is it preferable that the composition be devoid of a soap ingredient In addition, fragrances can also be added however they can have irritating results.

In some embodiments of the present invention the compound. In still other embodiments of the present invention the compound includes less than 1% of each of Sclerotium Gum, Sisymbrium Officinale Extract, Actium Lappa Root Extract, Citric Acid, Glycolic Acid, and Zinc PCA.

In some other embodiments of the present invention the compound includes various percentages of corundum #80, strawberry achenes (300-500 um) or Raspberry Seeds (300-500 um).

In some other embodiments of the present invention the compound includes a pumice element in an amount of about 25% to about 35% of the whole compound.

In addition, it is contemplated that the compound will be applied in linear motions preferably and removed from the skin with warm water after a maximum 60 seconds. Longer time periods can be used however, the time period will likely be subject to the sensitivity of the user. It has been found that the intended user, namely, an acne sufferer, is not apply any exfoliation compound for longer than 60 seconds they debilitating irritation and risk the rendering the exfoliation process ineffective and even harmful to the overall treatment regimen.

It will be apparent to one of skill in the art that described herein is a novel compound for exfoliating skin. While the invention has been described with reference to specific preferred embodiments, it is not limited to these embodiments. The invention may be modified or varied in many ways and such modifications and variations as would be obvious to one of skill in the art are within the scope and spirit of the invention and are included within the scope of the following claims.

1. A topical skin care exfoliating compound comprising;
a first subcompound formed from a clay based substance;
a second subcompound formed from an exfoliating substance selected from the group consisting of crystals, beads, polyethylene, nuts, natural rough products, corundum crystals, crushed almond shells and crushed apricot kernels.

2. The compound according to claim 1 further comprising a medicated ingredient, where the medicated ingredient is selected from the group consisting of an anti-acne medicine and an anti-bacteria medicine.

3. The compound according to claim 2 where the medicated ingredient includes about 10% sulphur by weight of the whole compound.

4. The compound according to claim 1 where the clay based substance includes at least one of the following deionized water, kaolin, bentonite, glycerin, zinc oxide, eucalyptus oil, propylene glycol, magnesium aluminum silicate, polysorbate-20, titanium dioxide, diazolidinyl urea, and propylparaben.

5. The compound according to claim 1 further comprising less than about 1% of each of Sclerotium Gum, Sisymbrium Officinale Extract, Actium Lappa Root Extract, Citric Acid, Glycolic Acid, and Zinc PCA.

6. The compound according to claim 1 further comprising at least one of corundum #80, strawberry achenes (300-500 um) and Raspberry Seeds (300-500 um).
